(19) United States
(12) Patent Application Publication
(10) Pub. No.: US 2024/0197534 A1
(43) Pub. Date: Jun. 20, 2024

(54) METHOD FOR PROVIDING CONTROL DATA FOR AN OPHTHALMOLOGICAL LASER FOR PROVIDING A TRANSITION ZONE BETWEEN INTERFACES

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/539,883

(22) Filed: Dec. 14, 2023

(30) Foreign Application Priority Data
Dec. 16, 2022 (DE) 10 2022 133 644.1

(51) Int. Cl. A61F 9/008

(52) U.S. Cl. CPC A61F 9/00825; A61B 2018/00904

(57) ABSTRACT

The invention relates to a method for providing control data for an ophthalmological laser (12) of a treatment apparatus (10). As steps, the method includes determining an anterior (14) and a posterior (16) interface in a cornea (17) of a human or animal eye; determining a transition zone, in which the interfaces (14, 16) are connected to each other, wherein transition positions (24) are determined on the anterior interface (14), from which an incision progression of the anterior interface (14) is changed towards the posterior interface (16), and wherein transition positions (26) are determined on the posterior interface (16), at which an incision progression of the posterior interface (16) is changed towards the anterior interface (14), such that the respective incision progressions of the interfaces (14, 16) converge and connect to each other in the transition zone; and providing control data for controlling the ophthalmological laser (12), which includes the interfaces (14, 16) and the incision progression of the transition zone.

### FIELD

The invention relates to a method for providing control data for an ophthalmological laser of a treatment apparatus, to a control device, which is configured to perform the method, to a treatment apparatus with such a control device, to a computer program and to a computer-readable medium, on which the computer program is stored.

### BACKGROUND

Treatment apparatuses and methods for controlling lasers, which are for example employed for the correction of an optical visual disorder of a cornea, are known per se in the art. For example, a pulsed laser and a beam focusing device can be formed such that laser beam spots cause an optical breakthrough, in particular a photodisruption, in a focus situated within a tissue of the cornea on a respectively preset incision surface. Usually, an anterior and a posterior interface are defined, which delimit the volume or the lenticule, and which can then be removed from a cornea, wherein a principal correction portion is performed in an optical zone. A transition zone adjoins to this optical zone, by which the interfaces are connected. Although it is assumed that the transition zone does not have an optical influence, empirical values indicate that this is nevertheless the case.

### SUMMARY

Therefore, it is an object of the invention to provide improved incision progressions for anterior and posterior interfaces for a volume body to be extracted, in particular in a transition zone.

This object is solved by the independent claims. Advantageous embodiments of the invention are disclosed in the dependent claims, in the following description as well as in the figures.

The invention is based on the idea that, departing from previous transition zone concepts, not only one of the interfaces is adapted to converge to the other one, but the incision progression or the curvature of both interfaces converges towards each other to progressively approach each other and to reduce a distance between the interfaces to zero.

By the invention, a method for providing control data for an ophthalmological laser of a treatment apparatus is provided, wherein the method includes the following steps performed by a control device. Determining an anterior and a posterior interface in a cornea of a human or animal eye, determining a transition zone, in which the interfaces are connected to each other, wherein transition positions are determined on the anterior interface, from which an incision progression on the anterior interface is changed towards the posterior interface, and wherein transition positions are determined on the posterior interface, from which an incision progression of the posterior interface is changed towards the anterior interface such that the respective incision progressions of the interfaces converge and connect to each other in the transition zone. Finally, the method includes providing control data for controlling the ophthalmological laser, which includes the interfaces and the incision progression of the transition zone.

In other words, interfaces for a volume body can first be determined, which causes a correction of the cornea after removal. These anterior and posterior interfaces can in particular specify a correction in an optical zone, wherein a connection between these interfaces is additionally planned, which is situated in a transition zone.

Therefore, transition positions are determined on the respective interface, which in particular can specify a point in a radial direction, from which the incision progression or a curvature differs from the original curvature used in the optical zone. This means, the transition positions set the beginning of the transition zone. In particular, the respective incision progression or curvature progression from the respective transition position can experience a severe curvature change or direction change such that the interfaces each converge to each other from the transition positions. This means, the incision progressions can progressively approach each other.

The transition positions of the anterior interface and the transition surface of the posterior interface do not have to be located at one level viewed in the radial direction, but can differ from each other in the radial direction, wherein the radially viewed innermost transition positions thus determine the optical zone and can determine the diameter of the volume body to be removed in the positions, in which the incision progressions concur or meet in the transition zone.

The control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

By the invention, the advantage arises that by the illustrated modeling of the transition zone, an improved stability of the cornea can be achieved and the development of vision restricting effects, in particular higher order aberrations, can additionally be reduced or avoided. In addition, the probability of an under-correction of the cornea is reduced since less hard edges are present upon collapse of the corneal layers after removing the volume body, whereby an epithelial layer can better adapt.

The invention also includes further embodiments, by which additional advantages arise.

In a further embodiment it is provided that the respective incision progression includes sections with different slope values. In other words, a slope or curvature can change in the respective incision progression, whereby the respective interface thus can have at least two different slope values from the transition position in the transition zone. Thus, at least one of the interfaces includes at least one further point in the transition zone after the transition position, which provides an induced gradient in multiple smaller zones with differing slope values. Hereby, a suitable configuration for modeling the transition zone can be provided.

In a further embodiment it is provided that a pose of the transition positions of the posterior interface differs from a pose of the transition positions of the anterior interface viewed in the radial direction. In other words, non-corresponding positions of the interface points or transition positions can be provided in radial direction, or different radial distances of the transition positions of the respective interface. Thus, the transition positions of the respective interface are not one above the other or one behind the other viewed from the direction of the optical axis. This means that the incision progression of the respective interface changes at different radial distances such that they converge towards each other.

Preferably, it is provided that the transition positions of the posterior interface are set further inward in the radial direction than the transition positions of the anterior interface. In other words, viewed in the radial direction, the incision progressions of the posterior interface are earlier changed towards the anterior interface than those of the anterior interface. Thus, a slope of the anterior interface in the transition zone can for example be lower than that of the anterior interface.

In a further embodiment it is provided that the incision progressions of the anterior and the posterior interface converge to each other with different slope magnitudes in the transition zone. This means that the incision progressions do not symmetrically converge to each other, but at different speeds. Therein, the absolute value of the slope is meant by the slope magnitude.

In a further embodiment it is provided that the incision progression of the respective interface in the transition zone is continuous and differentiable. In particular, it can be provided that hard edges are not present in the transition positions. For example, a straight connection line is not to be provided, which directly connects the respective interfaces or transition positions to each other.

In a further embodiment it is provided that the incision progressions of the respective interfaces converge to each other in the transition zone such that they meet halfway. This means that each of the incision progressions has a portion of 50 percent towards the respectively other interface before they connect to each other.

A further aspect of the invention relates to a method for controlling a treatment apparatus. Therein, the method includes method steps of at least one embodiment of a method as described above. Furthermore, the method for controlling the treatment apparatus also includes the step of transferring the provided control data to at least one ophthalmological laser of the treatment apparatus. The treatment apparatus, in particular the laser, can then be controlled with the control data for correcting a visual disorder.

The respective method can include at least one additional step, which is executed if and only if an application case or an application situation occurs, which was not explicitly described here. The step can for example include outputting an error message and/or outputting a request for inputting a user feedback. Additionally or alternatively, it can be provided that a default setting and/or a predetermined initial state are set.

A further aspect of the invention relates to a control device, which is formed to perform the steps of at least one embodiment of one or both of the previously described methods. The control device can include a computing unit for electronic data processing such as for example a processor. The computing unit can include at least one microcontroller and/or at least one microprocessor. The computing unit can be designed as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by means of the computing unit, whereby the control device is caused to execute the respective embodiment. The control device can be formed as a control chip or control unit. The control device can for example be included by a computer or computer network.

A further aspect of the invention relates to a treatment apparatus with at least one ophthalmological laser and a control device, which is formed to perform the steps of at least one embodiment of one or both of the previously described methods. The respective ophthalmological laser can be formed to at least partially separate a predefined corneal volume with predefined interfaces of a human or animal eye by means of photodisruption or ablation.

In a further advantageous configuration of the treatment apparatus according to the invention, the laser can be suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 kilohertz (kHz), preferably between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in laser technology. Thereby, it is advantageously avoided that an unintended damage to the cornea is caused by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy is allowed in the generation of the interfaces. In particular, a range between 700 nm and 780 nm can also be selected as the wavelength range.

In further advantageous embodiments of the treatment apparatus according to the invention, the control device can include at least one storage device for at least temporary storage of at least one control dataset, wherein the control dataset include(s) control data for positioning and/or for focusing individual laser pulses in the cornea; and can include at least one beam device for beam guidance and/or beam shaping and/or beam deflection and/or beam focusing of a laser beam of the laser.

A further aspect of the invention relates to a computer program. The computer program includes commands, which for example form a program code. The program code can include at least one control dataset with the respective control data for the respective laser. Upon execution of the program code by means of a computer or a computer network, it is caused to execute the previously described method or at least an embodiment thereof.

A further aspect of the invention relates to a computer-readable medium (storage medium), on which the above mentioned computer program and the commands thereof, respectively, are stored. For executing the computer program, a computer or a computer network can access the computer-readable medium and read out the content thereof. The storage medium is for example formed as a data memory, in particular at least partially as a volatile or non-volatile data memory. A non-volatile data memory can be a flash memory and/or an SSD (solid state drive) and/or a hard disk. A volatile data memory can be a RAM (random access memory). The commands can for example be present as a source code of a programming language and/or as assembler and/or binary code.

Further features and advantages of one of the described aspects of the invention can result from embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not include all of the features of an originally formulated claim or extend beyond or deviate from the feature combinations set forth in the relations of the claims.

FIG. 1 depicts a schematic representation of a treatment apparatus according to an exemplary embodiment.

FIG. 2a depicts a schematic incision progression of interfaces according to the prior art.

FIG. 2b depicts a schematic incision progression of interfaces according to an exemplary embodiment.

In the figures, identical or functionally identical elements are provided with the same reference signs.

### DETAILED DESCRIPTION

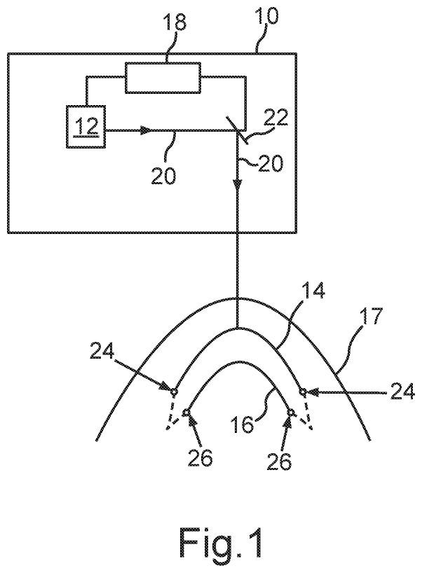

FIG. 1 shows a schematic representation of a treatment apparatus 10 with an eye surgical laser 12 for the separation of a lenticule defined by control data from a cornea 17 for example by means of photodisruption and/or ablation. Thereby, a visual disorder can for example be corrected.

FIG. 1 depicts the cornea 17 in a cross-section, thus a side view, in a plane, which extends axially to the optical axis of the eye. For separating the lenticule, an anterior interface 14 and a posterior interface 16 of a lenticule are provided in the control data, on which a cavitation bubble path for separating the lenticule from the cornea 17 can be generated. These interfaces 14, 16 form incision surfaces, along which an incision for separating the lenticule can be performed. One recognizes that a control device 18 for the laser 12 can be formed besides the laser 12, such that it can emit pulsed laser pulses for example in a predefined pattern for generating the interfaces 14, 16. Alternatively, the control device 18 can be a control device 18 external with respect to the treatment apparatus 10.

Furthermore, FIG. 1 shows that the laser beam 20 generated by the laser 12 is deflected towards the cornea 17 by means of a beam device 22, namely a beam deflection device such as for example a rotation scanner. The beam deflection device 22 is also controlled by the control device 18 to generate the interfaces 14, 16.

The illustrated laser 12 can preferably be a photodisruptive and/or ablative laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz. Optionally, the control device 18 additionally includes a storage device (not illustrated) for at least temporary storage of at least one control dataset, wherein the control dataset or datasets include(s) control data for positioning and/or for focusing individual laser pulses in the cornea. The position data and/or focusing data of the individual laser pulses, that is the lenticule geometry of the lenticule to be separated, is generated based on predetermined control data, in particular from a previously measured topography and/or pachymetry and/or the morphology of the cornea or of the optical visual disorder correction to be generated.

The control device 18 can be additionally formed to determine the anterior interface 14 and the posterior interface 16, in particular an incision progression in the cornea 17. Furthermore, a transition zone can be planned, in which the interfaces 14, 16 connect to each other, to thus provide a closed lenticule, which can be subsequently removed from the cornea 17. Hereto, transition positions 24 of the anterior interface 14 and transition positions 26 of the posterior interface 16 can be determined in the transition zone, at which an incision progression of the respective interfaces 14, 16 changes.

Thus, it is provided that an incision progression of the anterior interface 14 changes at the transition position 24 towards the posterior interface 16 and an incision progression of the posterior interface 16 additionally changes from the transition position 26 towards the anterior interface 14. This means, the incision progressions of the interfaces 14, 16 converge towards each other in the transition zone starting at the respective transition position 24, 26 and thus connect the interfaces 14, 16. This convergence of the incision progressions in the transition zone is only schematically illustrated here, wherein the respective incision progressions can differ, in particular a curvature or slope. Furthermore, the slope values of the incision progressions can change in the transition zone, which means that the respective incision progressions can include zones with different slope values.

Preferably, it is provided that the transition positions 26 of the posterior interface 16 are further in the center of the cornea 17 viewed in the radial direction than the transition positions 24 of the anterior interface 14. Thus, an optical zone for the treatment is set based on the transition positions 26, which are more centrally situated. Furthermore, it can preferably be provided that the incision progression is continuous and differentiable starting from the transition positions 24, 26 until concurrence (e.g., where the incisions meet), which means that a hard edge does not occur at the respective transition position 24, 26, in particular not a direct connection between the respective transition positions 24, 26. Thus, it can be achieved that the incision progressions progressively approach each other in the transition zone and thus connect the interfaces 14, 16, which results in better treatment results, in particular a reduction of higher order aberrations.

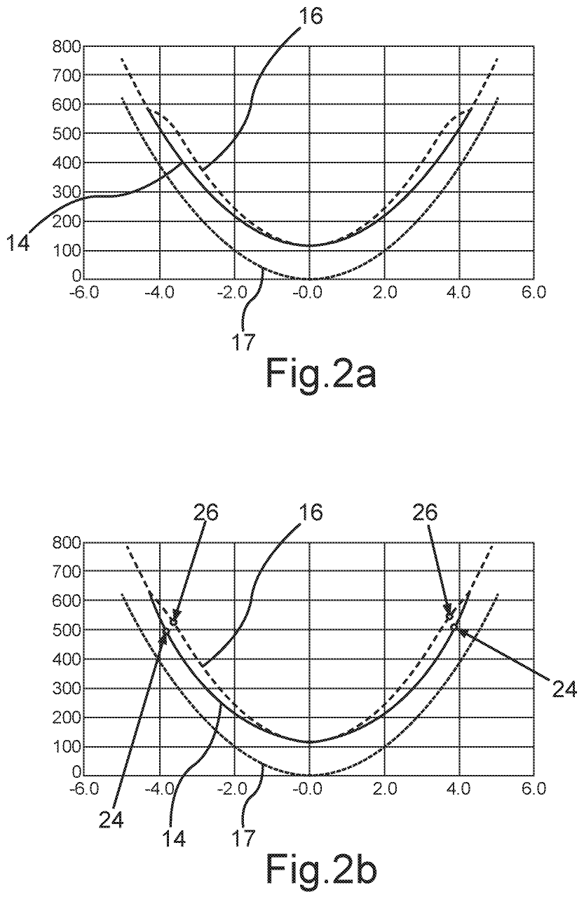

In FIGS. 2a and 2b, schematic diagrams are shown, which each show a cornea 17 with an anterior interface 14 and a posterior interface 16. Herein, FIG. 2a shows incision progressions of the interfaces 14, 16 as known in the art. In previous incision progressions, it can be provided that one of the two interfaces, in this case the posterior interface 16, changes an incision progression in the transition zone and extends towards the anterior interface 14 to connect to it and to form a volume body. Herein, a progression of the anterior interface 14 is not changed.

In FIG. 2b, the incision progressions according to an exemplary embodiment are illustrated. Herein, both interfaces can converge to each other in the transition zone to connect to each other. In particular, it is apparent in FIG. 2b that the incision progressions change at the transition positions 24, 26 such that they converge to each other and thus a smoother transition arises between the interfaces 14, 16.

Such an incision type is particularly advantageous for hyperopia correction and/or a mixed astigmatism, wherein these incision progressions are not only restricted to these treatments, but can also be applied to all further treatment methods, in which a lenticule is removed.

Overall, the examples show how an improved incision progression can be provided in the transition zone.

1. A method for providing control data for an ophthalmological laser of a treatment apparatus, wherein the method comprises the following steps performed by a control device:
determining an anterior interface and a posterior interface in a cornea of a human or animal eye;
determining a transition zone, in which the anterior interfaces and the posterior interface are connected to each other, wherein transition positions are determined on the anterior interface, from which an incision progression of the anterior interface is changed towards the posterior interface, and wherein transition positions are determined on the posterior interface, from which an incision progression of the posterior interface is changed towards the anterior interface, such that the respective incision progressions of the interfaces converge and connect to each other in the transition zone; and
providing control data for controlling the ophthalmological laser, which includes the interfaces and the incision progression of the transition zone.

2. The method according to claim 1, wherein the respective incision progression comprises sections with different slope values.

3. The method according to claim 1, wherein a pose of the transition positions of the posterior interface differs from a pose of the transition positions of the anterior interface viewed in a radial direction.

4. The method according to claim 3, wherein the transition positions of the posterior interface are set further inward in the radial direction than the transition positions of the anterior interface.

5. The method according to claim 1, wherein the incision progressions of the anterior and the posterior interface converge towards each other with different slope magnitudes in the transition zone.

6. The method according to claim 1, wherein the incision progression of the respective interface is continuous and differentiable in the transition zone.

7. The method according to claim 1, wherein the incision progressions of the respective interfaces converge towards each other in the transition zone such that they meet halfway.

8. A method for controlling a treatment apparatus, wherein the method comprises the following steps:
the method steps of the method according to claim 1, and
transferring the provided control data to a respective ophthalmological laser of the treatment apparatus.

9. A control device, which is configured to perform the method according to claim 1.

10. A treatment apparatus with at least one ophthalmological laser for separating a corneal volume with predefined interfaces of a human or animal eye by means of photodisruption or ablation and at least one control device according to claim 9.

11. The treatment apparatus according to claim 10, wherein the laser is formed to emit laser pulses in a wavelength range between 300 nm and 1400 nm at a respective pulse duration between 1 fs and 1 ns and a repetition frequency of greater than 10 kHz.

12. The treatment apparatus according to claim 10, wherein the control device comprises at least one storage device for at least temporary storage of at least one control dataset, wherein the at least one control dataset includes control data for positioning and/or focusing individual laser pulses in the eye; and
wherein the treatment apparatus includes at least one beam deflection device for beam guidance and/or beam shaping and/or beam deflection and/or beam focusing of a laser beam of the laser.

13. A non-transitory computer-readable medium, on which a computer program is stored, the computer program comprising commands that cause a control device to execute the method according to claim 1.

14. (canceled)
